(19) United States
(12) Patent Application Publication
(10) Pub. No.: US 2023/0364001 A1
(43) Pub. Date: Nov. 16, 2023

(54) TOOTH ANTI-STAINING COMPOSITION, KIT AND METHOD OF USING THE SAME

(71) Applicant: TOOTHFILM INC., Taipei City (TW)

(72) Inventors: CHUNG-LIN LEE, Taipei City (TW); ZHI-YUN LIN, Taipei City (TW)

(21) Appl. No.: 17/987,336

(22) Filed: Nov. 15, 2022

(30) Foreign Application Priority Data
May 12, 2022 (TW) 111117925

(51) Int. Cl. A61K 8/87; A61K 8/24; A61K 8/04; A61K 8/19; A61K 8/92; A61K 8/81; A61Q 11/02

(52) U.S. Cl. CPC A61K 8/87; A61K 8/24; A61K 8/042; A61K 8/19; A61K 8/922; A61K 8/8182; A61Q 11/02; A61K 2800/28

(57) ABSTRACT

The present disclosure relates to a tooth anti-staining composition comprising polyurethane and dicalcium phosphate and optionally other orally acceptable carriers and/or excipients. The present disclosure also provides a tooth anti-staining kit and a tooth anti-staining method with the tooth anti-staining composition.

### CROSS REFERENCE TO RELATED APPLICATIONS

The non-provisional patent application claims priority to Taiwan patent application with serial number TW111117925 filed on May 12, 2022. This and all other extrinsic materials discussed herein are incorporated by reference in their entirety.

### BACKGROUND

### Technology Field

The disclosure relates to a tooth anti-staining composition, kit and method of using the same.

### Description of Related Art

Recently, more and more people pay attention to the beauty of their teeth. White and bright teeth are what everyone wants to have, and tooth bleaching is one of the quick and effective methods. There are many causes of tooth discoloration, mainly divided into “physiological” and “pathological” causes.

The “physiological” cause of tooth discoloration is primarily age-related. Teeth take on a yellowish tint with age. In addition, dietary habits, such as drinking tea, coffee, red wine or habitually eating curry, may cause food pigments to deposit on the surface of the teeth, thereby accelerating the phenomenon of yellow teeth. The “pathological” cause of tooth discoloration is commonly found in tooth decay. The teeth after the root canal therapy or dental trauma can easily cause local discoloration of the tooth.

Tooth bleaching is the most effective way to remove pigment deposited on teeth, and its effect depends on the concentration of the peroxide and how long the peroxide is in contact with the teeth. The dietary habits during the treatment will affect the overall whitening effect. At present, many studies have pointed out that after tooth bleaching, it can cause damage to the surface of the teeth and result in the deposition of pigments on the teeth more easily, and it is more likely to cause tooth sensitivity. Especially, drinking coffee, cola or other after a tooth bleaching procedure can cause teeth to stain more easily. Therefore, to solve the above problems, there is still a need for products and methods for prolonging the whitening efficacy after tooth whitening and preventing pigment reattachment to the teeth.

### SUMMARY

The present disclosure provides a tooth anti-staining composition, wherein the composition comprises polyurethane and dicalcium phosphate, and optionally other orally acceptable carriers and/or excipients.

The present disclosure provides a tooth anti-staining kit, which comprises a tooth anti-staining composition and an instruction for using the tooth anti-staining composition.

The present disclosure provides a method for anti-staining of teeth, which comprises applying a tooth anti-staining composition on the dental surfaces, wherein the tooth anti-staining composition comprises polyurethane and dicalcium phosphate and optionally other orally acceptable carriers and/or excipients.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a tooth color chart of shade guide made by using a camera (Canon M6 Mark ii) with reference to the whitening shade guide (VITA Bleachedguide 3D-MASTER).

FIG. 2 shows the shade change of the groups of the anti-staining gel formulation comprising different concentrations of polyurethane-35.

FIG. 3 shows the shade change of the groups of the anti-staining gel formulation comprising different concentrations of dicalcium phosphate.

FIG. 4 shows the shade change of the groups of the anti-staining gel formulation comprising different concentrations of calcium lactate.

FIGS. 5, 6-1, 6-2, 6-3, 7, 8 and 9 show the shade change of the groups of the anti-staining gel formulation comprising different concentrations of polyurethane-35, dicalcium phosphate and calcium lactate.

FIG. 10 shows the shade change of tooth whitening effect.

FIG. 11 shows the shade change of anti-staining effect of teeth.

### DETAILED DESCRIPTION OF THE DISCLOSURE

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details.

### Definitions

As used herein, each of the following terms has the meaning associated with it in this section.

Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Furthermore, two or more steps or actions may or may not be performed simultaneously.

As used herein, the articles “a” and “an” refer to one or to more than one (i.e., to at least one) of the grammatical object of the article. By way of example, “an element” means one element or more than one element.

All percentages used herein are by weight of the tooth anti-staining composition, unless otherwise specified. The ratios used herein are weight ratios of the respective components, unless otherwise specified. All measurements are performed at 25° C., unless otherwise specified.

As used herein, the term “tooth” or “teeth” refers to a natural tooth or an artificial dentition. As used herein, “dental surface” refers to a surface of a natural tooth or a hard surface of artificial dentition including a denture, dental cap, crown, plate, bridge, filling, dental implant and the like. In one embodiment, the dental surface refers to a surface of a natural tooth.

As used herein, the term “composition” refers to a mixture of at least one compound useful in the present disclosure and orally acceptable carriers and/or excipients.

As used herein, the term “orally acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “effective amount” means an amount of a compound or composition sufficient to significantly induce a positive benefit, preferably an oral health benefit, but low enough to avoid serious side effects, i.e., to provide a reasonable benefit to risk ratio, within the sound judgment of a skilled artisan.

As used herein, the term “synergism” or “synergistic effect” means that two or more ingredients produce an effect that is greater than the simple summation of the same effect produced by administration of each of the ingredients. This effect is for example in prolonging the anti-staining effect of teeth.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

### Tooth Anti-staining Composition

As used herein, a “tooth anti-staining composition” refers to a composition for which the intended use can include oral care, oral hygiene, or oral appearance. The tooth anti-staining composition of the present disclosure comprises polyurethane and dicalcium phosphate and optionally other orally acceptable carriers and/or excipients.

The tooth anti-staining composition of the present disclosure may comprise orally acceptable amount of polyurethane. In one embodiment, the tooth anti-staining composition of the present disclosure may comprise polyurethane in an amount of from about 30% to about 3% by total weight of the composition, preferably, from about 30% to about 10%, and more preferably, from about 30% to about 20%. In another embodiment, the amount of polyurethane in the tooth anti-staining composition of the present disclosure may be 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4% or 3% by total weight of the composition, or any content between the above-mentioned contents.

The tooth anti-staining composition of the present disclosure may comprise orally acceptable amount of dicalcium phosphate. In one embodiment, the tooth anti-staining composition of the present disclosure may comprise dicalcium phosphate in an amount of from about 25% to about 0.1% by total weight of the composition, preferably, from about 10% to about 0.5%, more preferably, from about 5% to about 1%, and the mast preferably, from about 3% to about 1%. In another embodiment, the amount of dicalcium phosphat in the tooth anti-staining composition of the present disclosure may be 25%, 20%, 15%, 10%, 9%, 8%, 7%, 6%, 5%, 4%, 3%, 2.8%, 2.6%, 2.4%, 2.2%, 2.0%, 1.8%, 1.6%, 1.4%, 1.2%, 1.0%, 0.9%, 0.8%, 0.7%, 0.6%, 0.5%, 0.4%, 0.3%, 0.2% or 0.1% by total weight of the composition, or any content between the above-mentioned contents.

The tooth anti-staining composition of the present disclosure may comprise orally acceptable amount of calcium lactate. In one embodiment, the tooth anti-staining composition may comprise calcium lactate in an amount of from about 25% to about 0.1% by total weight of the composition, preferably, from about 10% to about 0.1%, and more preferably, from about 5% to about 0.5%, and the most preferably, from about 3% to about 0.5%. In another embodiment, the amount of calcium lactate in the tooth anti-staining composition may be 25%, 20%, 15%, 10%, 9%, 8%, 7%, 6%, 5%, 4%, 3%, 2%, 1.8%, 1.6%, 1.4%, 1.2%, 1.0%, 0.9%, 0.8%, 0.7%, 0.6%, 0.5%, 0.4%, 0.3%, 0.2% or 0.1% by total weight of the composition, or any content between the above-mentioned contents.

The tooth anti-staining composition of the present disclosure may comprise at least one thickener of crosslinked polyvinylpyrrolidone. The crosslinked polyvinylpyrrolidone may include homopolymers of polyvinylpyrrolidone or N-vinyl-2-pyrrolidone. In one embodiment, the tooth anti-staining composition may comprise polyvinylpyrrolidone in an amount of from about 30% to about 2.5% by total weight of the composition, preferably, from about 15% to about 10%.

The tooth anti-staining composition of the present disclosure may comprise polyphosphate materials or synthetic anionic polymers as anti-calculus agents, such as, copolymers of polyacrylates, maleic anhydride and/or methyl vinyl ether and the mixture thereof, which are known to be effective in reducing calcium phosphate mineral deposition related to calculus formation. Preferably, the anti-calculus agent is acrylates copolymer ○ In one embodiment, the amount of acrylates copolymer in the tooth anti-staining composition may be in an amount of from about 20% to about 0.2% by total weight of the composition, preferably, from about 2.5% to about 1.5%.

The tooth anti-staining composition of the present disclosure may comprise titanium dioxide. In one embodiment, the amount of titanium dioxide in the tooth anti-staining composition may be in an amount of from about 25% to about 0.1% by total weight of the composition, preferably, from about 5% to about 0.25%, and more preferably, from about 1.5% to about 0.5%.

### Tooth Whitening Composition

The present disclosure provides a tooth whitening composition comprising hydrogen peroxide and an orally acceptable carriers, wherein the tooth whitening composition may comprise hydrogen peroxide in an amount of from about of 0.1% to about 6% by total weight of the composition, preferably, from about 2% to about 6%, and more preferably, from about 4% to about 6%.

In one embodiment, the tooth whitening composition of the present disclosure further comprises one or more materials selected from the group consisting of water, glycerol, propylene glycol, potassium citrate, polyvinylpyrrolidone, acrylate, alkyl acrylate cross-linked polymers, potassium nitrate, ethyl menthane carboxamide, pearl extracts and other orally acceptable components.

### Carriers and Excipients

The “orally acceptable carriers” and “orally acceptable excipients” are used interchangeably herein, and refer to any carriers and/or excipients useful for the tooth anti-staining composition of the present disclosure. The orally acceptable carriers and/or excipients are not harmful to a mammal when retained in the mouth in amounts disclosed herein without swallowing for a period sufficient to permit effective contact with a dental surface as required herein. Generally, the orally acceptable carriers and/or excipients are not harmful even if unintentionally swallowed.

As used herein, the orally acceptable carriers and/or excipients mean any safe and effective materials that may be used in the tooth anti-staining composition in the embodiment or examples disclosed herein and do not alter the efficacy or properties of the main ingredients described above. The suitable orally acceptable carriers and/or excipients useful in the tooth anti-staining composition of the present disclosure may contain one or more materials selected from the group consisting of water, humectant, surfactant, anti-sensitizer, anti-bacterial and preservative, flavoring agent and the like.

The tooth anti-staining composition of the present disclosure may comprise at least one humectant selected from the group consisting of sorbite, glycerol, xylitol, propylene glycol and the combination thereof.

The tooth anti-staining composition of the present disclosure may comprise at least one surfactant, which may be may be anionic, nonionic, amphoteric, zwitterionic, cationic, or mixtures thereof. The surfactant is preferably selected from the group consisting of polysorbate 20, polysorbate 40, polysorbate 60, polysorbate 65, polysorbate 80 and the combination thereof.

The tooth anti-staining composition of the present disclosure may comprise at least one potassium salt as an anti-sensitizer, wherein the potassium salt is selected from the group consisting of potassium citrate, potassium nitrate, potassium chloride and the combination thereof.

The tooth anti-staining composition of the present disclosure may comprise at least one anti-bacterial or preservative, wherein the anti-bacterial or preservative is selected from the group consisting of phenoxyethanol, benzoic acid, salicylic acid, p-hydroxybenzoic acid, methyl-p-hydroxybenzoate, chlorphenesin, ethylhexylglycerin and the combination thereof.

The tooth anti-staining composition of the present disclosure may comprise at least one flavoring agent, wherein the flavoring agent comprises essential oils and various flavoring aldehydes, esters, alcohols, wherein examples of the essential oils include oil extracts from lemon, lime, grapefruit, orange, peppermint, wintergreen, sassafras, clove, sage, spearmint, marjoram, and/or cinnamon. In one embodiment, the flavoring agent comprises acesulfame potassium, ethyl menthane carboxamide, menthol, carvone or anethole.

The tooth anti-staining composition of the present disclosure may comprise at least one abrasive, wherein the abrasive is selected from the group consisting of aluminum hydroxide, aluminum silicate, dicalcium phosphate, silica, calcium carbonate, bentonite and the combination thereof.

It will be obvious to one of skill in the art that some components contained in the tooth anti-staining composition of the present disclosure may perform multiple functions. Therefore, the identification of a compound as having one function herein is not meant to exclude its use for other functions in a particular composition. For example, a compound such as menthol can act both as a flavorant and an antiseptic agent, and polysorbate 20 can act as both a surfactant and as an emulsifier.

### Dosage Form

The tooth anti-staining composition of the present disclosure remains in the oral cavity for a period of time sufficient to contact substantially the entire tooth surface to achieve anti-staining activity. Therefore, the tooth anti-staining composition of the present disclosure may be in the form of a solution, slurry, gel, paste or ointment or any other dosage form. In one embodiment, the dosage form of the tooth anti-staining composition is a mouthwash. In another embodiment, the dosage form of the tooth anti-staining composition is an applying gel. In other embodiment, the dosage form of the tooth anti-staining composition is a toothpaste.

The tooth whitening composition of the present disclosure remains in the oral cavity for a period of time sufficient to contact substantially the entire tooth surface to achieve whitening activity. Therefore, the tooth whitening composition of the present disclosure may be in the form of a solution, slurry, gel, paste or ointment or any other dosage form. In one embodiment, the dosage form of the tooth whitening composition is a mouthwash. In another embodiment, the dosage form of the tooth whitening composition is an applying gel. In other embodiment, the dosage form of the tooth whitening composition is a toothpaste.

### Use and Method

When applying the tooth anti-staining composition of the present disclosure, the user only needs to apply the tooth anti-staining composition to the surface of a human tooth in order to achieve the desired effects in the desired area of the tooth, such as anti-staining, keeping teeth white, fresh breath, etc. The tooth anti-staining composition of the present disclosure may be safely applied to oral surfaces other than teeth, such as oral mucosal tissue or gums, without resulting in or causing irritation or discomfort. The tooth anti-staining composition of the present disclosure may be applied to the teeth by any means, preferably with a toothbrush or braces or by direct rinsing.

In one embodiment, when the tooth anti-staining composition of the present disclosure is a dosage form of gel, paste or ointment, an effective amount of the tooth anti-staining composition may be applied to the dental surface by a toothbrush, and the tooth surface can be contacted with the tooth anti-staining composition for a period of time, and then the tooth surface is rinsed and cleaned by gargling. In another embodiment, when the tooth anti-staining composition of the present disclosure is a dosage form of gel, paste or ointment, an effective amount of the tooth anti-staining composition may be filled in a braces and worn into teeth, and the dental surface can be contacted with the tooth anti-staining composition for a period of time, and then the tooth surface is rinsed and cleaned by gargling. Depending on the concentration of the active ingredient in the tooth anti-staining composition, the contact time of the tooth anti-staining composition with the dental surface may vary, for example, within 30 minutes, preferably, from 30 seconds to 25 minutes, more preferably, from 30 seconds to 10 minutes, and most preferably, 30 seconds to 3 minutes.

In one embodiment, when the tooth anti-staining composition of the present disclosure is a dosage form of solution or slurry, such as mouthwash form, the tooth anti-staining composition can be used directly, left in the mouth for a period of time, and then rinsed off the composition from the dental surface by gargling.

There is no limitation on the application time point of the tooth anti-staining composition of the present disclosure. For example, it may be applied after cleaning with a dentifrice on weekdays. Preferably, the tooth anti-staining composition of the present disclosure may be applied after a dental cleaning or tooth whitening procedure has been performed by a dentist ○ Alternatively, the tooth anti-staining composition of the present disclosure can be applied at home after whitening the teeth by using a tooth whitening agent. Within the orally acceptable range, there is no particular limitation on the number or frequency of applying the tooth anti-staining composition of the present disclosure. Preferably, it can be applied immediately after daily dentifrice cleaning, preferably 3 times a day, twice a day, once a day, once every 2 days, once every 3 days, once every 4 days, once every 5 days, once every 6 days, or once every week. Alternatively, it can be used as directed by a dentist.

### Kits

The present disclosure provides a tooth anti-staining kit comprising the tooth anti-staining composition disclosed above and an instruction for use. A user can apply the tooth anti-staining composition with reference to the instructions.

The present disclosure provides a tooth whitening and anti-staining kit comprising the tooth anti-staining composition and the tooth whitening composition disclosed above and an instruction for use. The user may refer to the instruction for use, first apply the tooth whitening composition to perform the tooth whitening step, and then apply the tooth anti-staining composition to perform the tooth anti-staining step.

The following examples further illustrate the preferred embodiment of the present disclosure. However, it should be understood that the present disclosure is not limited to the contents of the examples described below.

### EXAMPLES

### Materials and Methods

### A. Selection Criteria of Teeth for Testing

The teeth used in the examples are human permanent teeth. The teeth are permanent teeth extracted from adults over the age of 20 due to the need for correction or abnormal tooth position. The selection criteria are set as the tooth surface is not filled, the crown and root are allowed to be half or complete, and the root has no apical lesions. However, enamel hypoplasia, tooth deformation, tooth fragmentation, root apex lesions, congenital abnormal tooth color, and tooth staining caused by smoking or eating betel nut for more than 1 year are excluded.

### B. Preparation of Anti-staining Agents

Anti-staining formulations in the form of gels containing different concentrations of polyurethane, dicalcium phosphate and calcium lactate are prepared, and the detailed ingredients are shown in Table 1A, wherein the other ingredients and contents in each formulations are the same except for the contents of polyurethane, dicalcium phosphate and calcium lactate. X represents the weight change of polyurethane-35 in the examples. Y represents the weight change of dicalcium phosphate in the examples. Z represents the weight change of calcium lactate in the examples.

TABLE 1A
The components of tooth anti-staining formulations

| | Proportion (%) | | |
|---|---|---|---|
| Components | Formulation A | Formulation B | Formulation C |
| polyurethane-35 (Polyurethane-35) | X % | 15% | 15% |
| Polyvinylpyrrolidone | 12.50% | 12.50% | 12.50% |
| acrylates copolymer | 2.00% | 2.00% | 2.00% |
| dicalcium phosphate | 1% | Y % | '; 1% |
| titanium dioxide | 0.8% | 0.8% | 0.8% |
| calcium lactate | 0.5% | 0.5% | Z% |
| Other components: | adding to | adding to | adding to |
| water, sorbitol, glycerol, propylene glycol, | 100% | 100% | 100% |
| potassium citrate, Chlorphenesin , | | | |
| Phenoxyethanol, polysorbate 80, | | | |
| acesulfame potassium, ethyl menthane | | | |
| carboxamide, silica, calcium oxide, | | | |
| potassium hydroxide, ethylhexylglycerin | | | |

### C. Preparation of Whitening Agents

A whitening formulation is prepared in the form of a gel containing hydrogen peroxide at a concentration of 5.6%, and the detailed ingredients are shown in Table 1B.

TABLE 1B
The components of whitening formulation

| Components | Proportion (%) |
|---|---|
| hydrogen peroxide | 5.6% |
| Other components: | adding to 100% |
| water, glycerol, propylene glycol, potassium citrate, | |
| Polyvinylpyrrolidone, acrylate, alkyl acrylatecross-linked | |
| polymers, potassium nitrate, ethyl menthane | |
| carboxamide, pearl extracts | |

### D. Staining Solution

The staining solution used in the following examples is prepared by using commercially available Nespresso espresso black coffee. The use temperature of the staining solution is 37±2.5° C. The anti-staining tests performed in the various examples use the staining solution prepared from the same espresso black coffee and having the same concentration and the same components.

### E. Preparation of Chart of Shade Guide

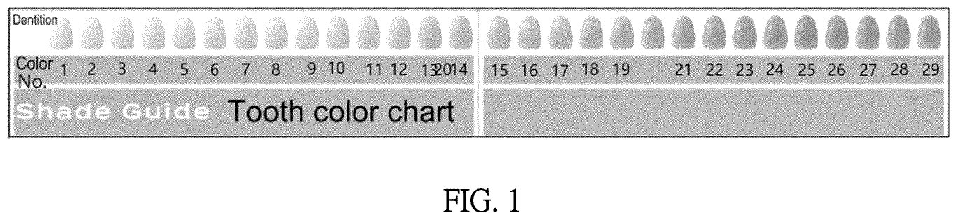

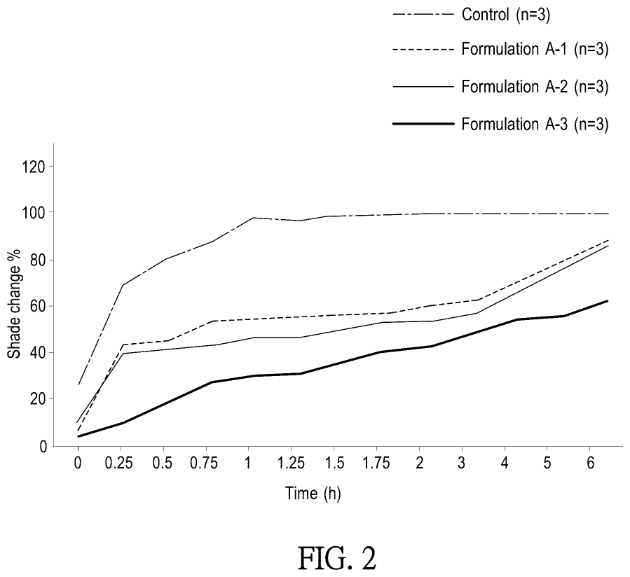

According to the 29-color whitening scale issued by the American Dental Association (ADA) as the colorimetric benchmark, using the whitening shade guide (VITA Bleachedguide 3D-MASTER) as the reference standard, a tooth color chart is made with a camera (Canon M6 Mark ii), as shown in FIG. 1, for comparing the results of tooth shade changes.

### Example 1

The teeth collected above were randomly selected and washed with 37% phosphoric acid, and then washed with water once to remove the dirt on the surface, and dried in an oven at 37° C.

The anti-staining gel formulations A containing different concentrations of polyurethane-35 were prepared, wherein Formulations A-1, A-2 and A-3 contained polyurethane-35 in an amount of 3%, 15% and 30% by total weight of the anti-staining gel formulations, respectively, as shown in Table 2, and the rest of the ingredients were the same, as shown in Table 1A. The teeth were soaked in the above anti-staining gel formulations for 15 minutes, then rinsed with primary water and dried, and the above steps were repeated 7 times. Then, it was soaked in the staining solution for 6 hours, and photographed every 15 minutes under the same conditions for the preparation of chart of shade guide as the above-mentioned.

TABLE 2

| Proportion (%) | Formulation A-1 | Formulation A-2 | Formulation A-3 |
|---|---|---|---|
| X | 3% | 15% | 30% |

The teeth of control group were treated with primary water instead of the anti-staining gel formulation A, and the same procedure as above was performed.

Results are as shown in FIG. 2. The average shade of the teeth of the control group before being soaked in the staining solution was 7.6, which increased to 28.33 after 1 hour, and the shade change was 97.7%. After 2 hours of continuous soaking, the teeth reached shade 29, and the shade change was 100%.

In experimental group, the average shade of the teeth of formulation A-1 group comprising 3% polyurethane-35 before being soaked in the staining solution was 2, which increased to 17.43 after 2 hours of continuous soaking, and the shade change was 60.11%. After 6 hours of continuous soaking, the average shade of the teeth reached 25.53, and the shade change was 88.04%. The average shade of the teeth of formulation A-2 group comprising 15% polyurethane-35 before being soaked in the staining solution was 3, which increased to 15.5 after 2 hours of continuous soaking, and the shade change was 53.44%. After 6 hours of continuous soaking, the average shade of the teeth reached 25, and the shade change was 86.2%. The average shade of the teeth of formulation A-3 group comprising 30% polyurethane-35 before being soaked in the staining solution was 1, which increased to 12.25 after 2 hours of continuous soaking, and the shade change was 42.24%. After 6 hours of continuous soaking, the average shade of the teeth reached 18, and the shade change was 62.06%.

As shown in the above results, in the control group without the treatment with the anti-stain gel of the present disclosure, the teeth had a weak anti-staining effect against coffee pigment, and the shade change reached 97.7% after soaking for one hour. The teeth treated with the anti-staining gel formulations A-1, A-2 and A-3 of the present disclosure can effectively improve the staining resistance of the teeth in the coffee environment for a long time compared with the control group.

### Example 2

The anti-staining gel formulations B containing different concentrations of dicalcium phosphate were prepared, wherein Formulations B-1, B-2 and B-3 contained dicalcium phosphate in an amount of 0%, 1% and 2% by total weight of the anti-staining gel formulations, respectively, as shown in Table 3, and the rest of the ingredients were the same, as shown in Table 1A. The teeth were soaked in the above anti-staining gel formulations for 15 minutes, then rinsed with primary water and dried, and the above steps were repeated 7 times. Then, it was soaked in the staining solution for 6 hours, and photographed every 15 minutes under the same conditions for the preparation of chart of shade guide as the above-mentioned.

TABLE 3

| Component | formulation B-1 | formulation B-2 | formulation B-3 |
|---|---|---|---|
| Y | 0% | 1% | 2% |

The teeth of control group were treated with primary water instead of the anti-staining gel formulation B, and the same procedure as above was performed.

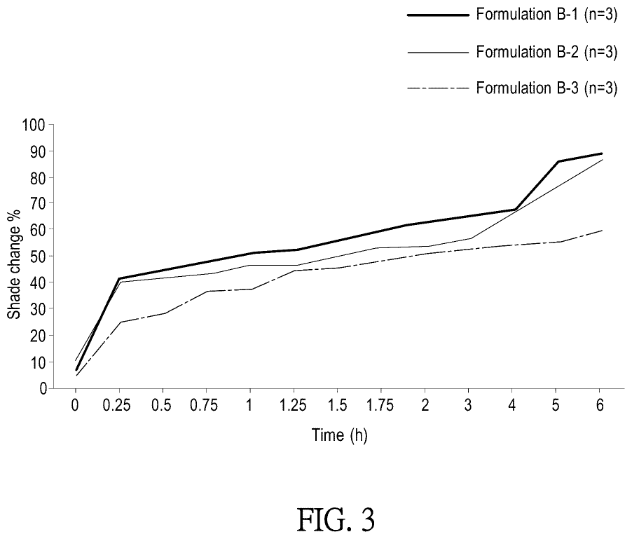

Results are as shown in FIG. 3. The average shade of the teeth of formulation B-1 group without dicalcium phosphate before being soaked in the staining solution was 2, which increased to 14.8 after 1 hour of continuous soaking, and the shade change was 51.03%, and increased to 18.3 after 2 hours of continuous soaking, and the shade change was 53.44%. After 6 hours of continuous soaking, the average shade of the teeth reached 25.7, and the shade change was 88.62%. The average shade of the teeth of formulation B-2 group comprising 1% dicalcium phosphate before being soaked in the staining solution was 3, which increased to 15.5 after 2 hours of continuous soaking, and the shade change was 53.44%. After 6 hours of continuous soaking, the average shade of the teeth reached 25, and the shade change was 86.2%. The average shade of the teeth of formulation B-3 group comprising 2% dicalcium phosphate before being soaked in the staining solution was 1.5, which increased to 14.85 after 2 hours of continuous soaking, and the shade change was 51.12%. After 6 hours of continuous soaking, the average shade of the teeth reached 17.22, and the shade change was 59.39%.

As described in Example 1, the shade change of the teeth of the control group without treatment with the anti-staining gel formulation of the present disclosure increased to 97.7% after soaking in coffee for 1 hour. As shown in the above results, the teeth treated with the anti-staining gel formulations without and with dicalcium phosphate showed better anti-staining effect compared to the control group. Notably, the anti-staining gel formulation comprising dicalcium phosphate can more effectively improve the staining resistance of teeth in the coffee environment for a long time. The anti-staining gel formulation comprising 2% dicalcium phosphate had better anti-staining effect than other concentrations.

### Example 3

The anti-staining gel formulations C containing different concentrations of calcium lactate were prepared, wherein Formulations C-1, C-2 and C-3 contained calcium lactate in an amount of 0%, 0.5% and 1% by total weight of the anti-staining gel formulations, respectively, as shown in Table 4, and the rest of the ingredients were the same, as shown in Table 1A. The teeth were soaked in the above anti-staining gel formulations for 15 minutes, then rinsed with primary water and dried, and the above steps were repeated 7 times. Then, it was soaked in the staining solution for 6 hours, and photographed every 15 minutes under the same conditions for the preparation of chart of shade guide as the above-mentioned.

TABLE 4

| Component | formulation C-1 | formulation C-2 | formulation C-3 |
|---|---|---|---|
| Z | 0% | 0.5% | 1% |

The teeth of control group were treated with primary water instead of the anti-staining gel formulation C, and the same procedure as above was performed.

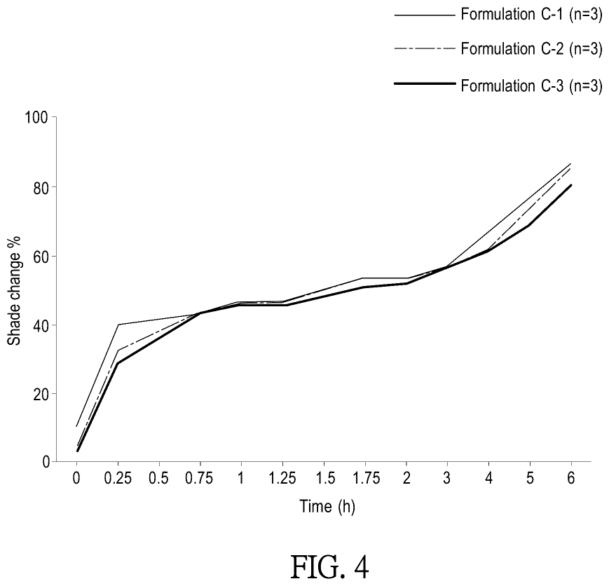

Results are as shown in FIG. 4. The average shade of the teeth of formulation C-1 group without calcium lactate before being soaked in the staining solution was 3, which increased to 13.5 after 1 hour of continuous soaking, and the shade change was 45.5%, and increased to 15.5 after 2 hours of continuous soaking, and the shade change was 53.44%. After 6 hours of continuous soaking, the average shade of the teeth reached 25, and the shade change was 86.2%. The average shade of the teeth of formulation C-2 group comprising 0.5% calcium lactate before being soaked in the staining solution was 1, which increased to 15.2 after 2 hours of continuous soaking, and the shade change was 52.41%. After 6 hours of continuous soaking, the average shade of the teeth reached 24.63, and the shade change was 84.94%. The average shade of the teeth of formulation C-3 group comprising 1% calcium lactate before being soaked in the staining solution was 1, which increased to 15.06 after 2 hours of continuous soaking, and the shade change was 51.95%. After 6 hours of continuous soaking, the average shade of the teeth reached 23.34, and the shade change was 80.34%.

As described in Example 1, the shade change of the teeth of the control group without treatment with the anti-staining gel formulation of the present disclosure increased to 97.7% after soaking in coffee for 1 hour. As shown in the above results, the teeth treated with the anti-staining gel formulations without and with calcium lactate showed better anti-staining effect compared to the control group. Notably, the anti-staining gel formulation comprising calcium lactate can more effectively improve the staining resistance of teeth in the coffee environment for a long time.

### Example 4

The anti-staining gel formulations D-1, D-2 and D-3 containing different concentrations of polyurethane-35, dicalcium phosphate and calcium lactate as shown in Table 5 were prepared respectively, and the rest of the ingredients were the same, as shown in Table 1A.

TABLE 5

| Component | formulation D-1 | formulation D-2 | formulation D-3 |
|---|---|---|---|
| X | 3% | 30% | 30% |
| Y | 0% | 2% | 2% |
| Z | 0% | 0% | 1% |

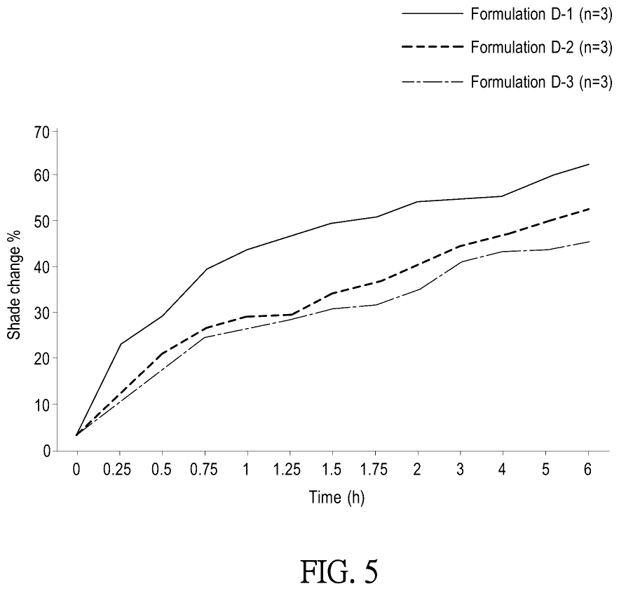

Results are as shown in FIG. 5. The average shade of the teeth of formulation D-1 group comprising 3% polyurethane-35, 0% dicalcium phosphate and 0% calcium lactate before being soaked in the staining solution was 1, which increased to 12.6 after 1 hour of continuous soaking, and the shade change was 43.6%, and increased to 15.6 after 2 hours of continuous soaking, and the shade change was 54.02%. After 6 hours of continuous soaking, the average shade of the teeth reached 18, and the shade change was 62.06%. The average shade of the teeth of formulation D-2 group comprising 30% polyurethane-35, 2% dicalcium phosphate and 0% calcium lactate before being soaked in the staining solution was 1, which increased to 11.6 after 2 hours of continuous soaking, and the shade change was 40.11%. After 6 hours of continuous soaking, the average shade of the teeth reached 15.1, and the shade change was 52.06%. The average shade of the teeth of formulation D-3 group comprising 30% polyurethane-35, 2% dicalcium phosphate and 1% calcium lactate before being soaked in the staining solution was 1, which increased to 10.1 after 2 hours of continuous soaking, and the shade change was 34.82%. After 6 hours of continuous soaking, the average shade of the teeth reached 13.1, and the shade change was 45.1%.

As shown in the above results, the anti-staining gel formulation D3 comprising polyurethane-35, dicalcium phosphate and calcium lactate was more able to improve the staining resistance of teeth in the coffee environment for a long time than the anti-staining gel formulation D1 comprising polyurethane-35 and the anti-staining gel formulation D2 comprising polyurethane-35 and dicalcium phosphate.

### Example 5

According to the above experiments, it is known that different concentrations of polyurethane-35, dicalcium phosphate and calcium lactate have certain staining resistance when used alone or in combination. The following examples further explore the synergistic effect of three different raw materials in the effect of component ratio and stain resistance.

The anti-staining gel formulations C-1, E, F and G containing different concentrations of polyurethane-35, dicalcium phosphate and calcium lactate as shown in Table 6 were formulated respectively, and the rest of the ingredients were the same, as shown in Table 1A.

TABLE 6

| | formulation | formulation | formulation | formulation |
|---|---|---|---|---|
| Components | C-1 | E | F | G |
| X | 15% | 15% | 0% | 0% |
| Y | 1% | 0% | 1% | 0% |
| Z | 0% | 0% | 0% | 0.5% |

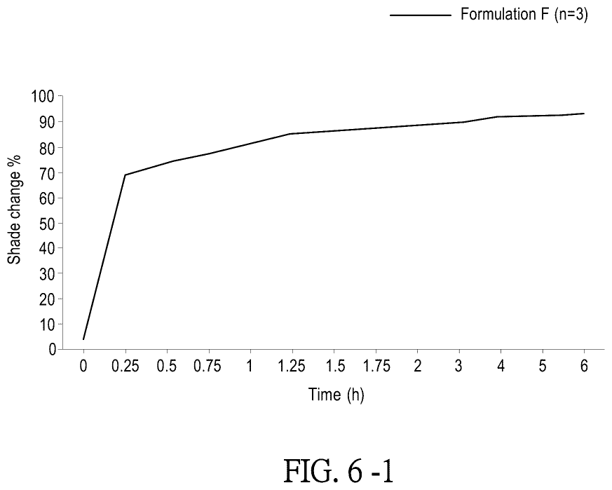

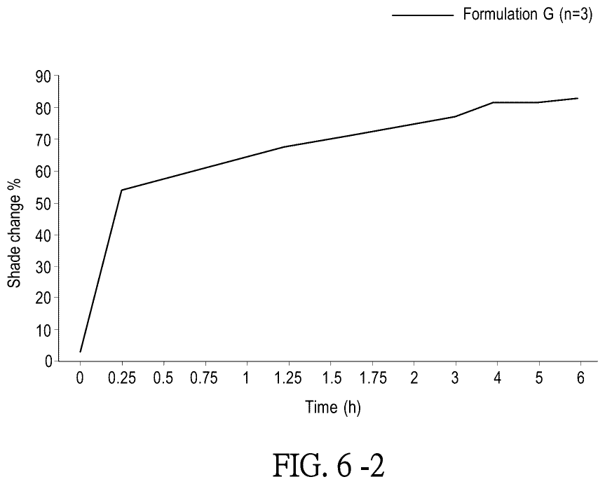

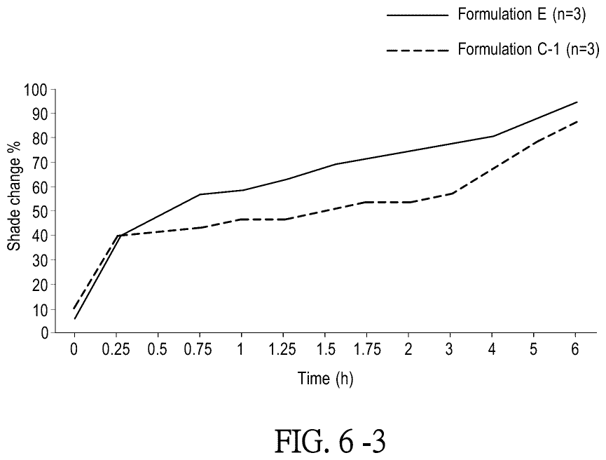

First, the anti-staining effect test of the anti-staining composition containing polyurethane-35, calcium hydrogen phosphate or calcium lactate alone were performed, and the results were shown in FIGS. 6-1 and 6-2. An synergistic effect test was performed by using polyurethane-35 and dicalcium phosphate, as the formulation C-1 and E in Table 6. The detailed anti-staining results were shown in FIG. 6-3.

The formulation C-1 was formulated according to Example 3 above, that is, the formulation C-1 comprised 15% polyurethane-35, 1% dicalcium phosphate and 0% calcium lactate. The average shade of the teeth of formulation C-1 group before being soaked in the staining solution was 3, which increased to 13.5 after 1 hour of continuous soaking, and the shade change was 45.5%, and increased to 15.5 after 2 hours of continuous soaking, and the shade change was 53.44%. After 6 hours of continuous soaking, the average shade of the teeth reached 25, and the shade change was 86.2%. The formulation E was formulated in the same way to comprised 15% polyurethane-35, 0% dicalcium phosphate and 0% calcium lactate. The average shade of the teeth of formulation E group before being soaked in the staining solution was 1.76, which increased to 17.06 after 1 hour of continuous soaking, and the shade change was 58.85%, and increased to 21.53 after 2 hours of continuous soaking, and the shade change was 74.25%. After 6 hours of continuous soaking, the average shade of the teeth reached 27.33, and the shade change was 94.25%. The above results showed that the formulation comprising polyurethane-35 in combination with dicalcium phosphate was more able to prolong the staining resistance of teeth than comprising polyurethane-35 alone.

### Example 6

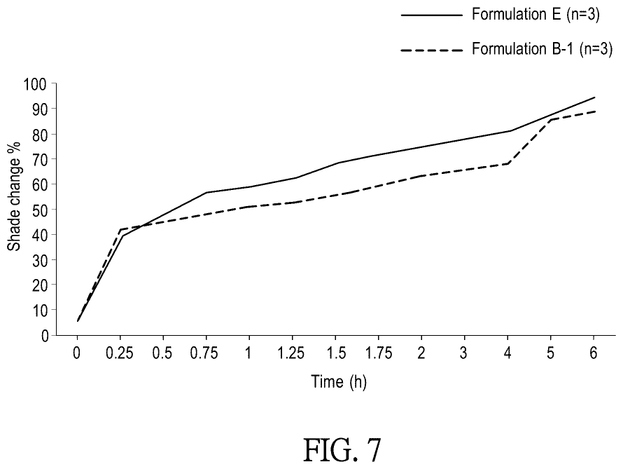

Example 6 was performed in a manner similar to Example 5 above. The anti-staining gel formulations B-1 and E containing different concentrations of polyurethane-35, dicalcium phosphate and calcium lactate as shown in Table 7 were formulated respectively, and the rest of the ingredients were the same, as shown in Table 1A. The detail results of staining resistance were shown in FIG. 7.

TABLE 7

| Components | formulation B-1 | formulation E |
|---|---|---|
| X | 15% | 15% |
| Y | 0% | 0% |
| Z | 0.5% | 0% |

The formulation B-1 was formulated according to Example 3 above, that is, the formulation B-1 comprised 15% polyurethane-35, 0% dicalcium phosphate and 0.5% calcium lactate. The average shade of the teeth of formulation B-1 group before being soaked in the staining solution was 2, which increased to 14.8 after 1 hour of continuous soaking, and the shade change was 51.03%, and increased to 18.3 after 2 hours of continuous soaking, and the shade change was 53.44%. After 6 hours of continuous soaking, the average shade of the teeth reached 25.7, and the shade change was 88.62%. The average shade of the teeth of formulation E group before being soaked in the staining solution was 1.76, which increased to 17.06 after 1 hour of continuous soaking, and the shade change was 58.85%, and increased to 21.53 after 2 hours of continuous soaking, and the shade change was 74.25%. After 6 hours of continuous soaking, the average shade of the teeth reached 27.33, and the shade change was 94.25%. The above results showed that the formulation comprising polyurethane-35 in combination with calcium lactate was more able to prolong the staining resistance of teeth than comprising polyurethane-35 alone.

### Example 7

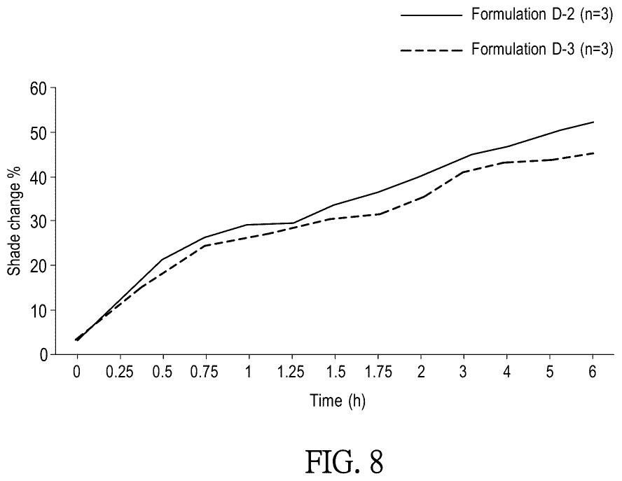

Example 7 was performed in a manner similar to Example 5 above. The anti-staining gel formulations D-2 and D-3 containing different concentrations of polyurethane-35, dicalcium phosphate and calcium lactate as shown in Table 8 were formulated respectively, and the rest of the ingredients were the same, as shown in Table 1A. The detail results of staining resistance were shown in FIG. 8.

TABLE 8

| Components | formulation D-2 | formulation D-3 |
|---|---|---|
| X | 30% | 30% |
| Y | 2% | 2% |
| Z | 0% | 1% |

The formulations D-2 and D-3 were formulated according to Example 4 above. The formulation D-2 comprised 30% polyurethane-35, 2% dicalcium phosphate and 0% calcium lactate. The average shade of the teeth of formulation D-2 group before being soaked in the staining solution was 1, which increased to 11.6 after 2 hours of continuous soaking, and the shade change was 40.11%, and increased to 15.1 after 6 hours of continuous soaking, and the shade change was 52.06%. The formulation D-3 comprised 30% polyurethane-35, 2% dicalcium phosphate and 1% calcium lactate. The average shade of the teeth of formulation D-3 group before being soaked in the staining solution was 1, which increased to 10.1 after 2 hours of continuous soaking, and the shade change was 34.82%, and increased to 13.1 after 6 hours of continuous soaking, and the shade change was 45.1%. The above results showed that the formulation comprising polyurethane-35 and dicalcium phosphat in combination with calcium lactate was more able to prolong the staining resistance of teeth than the formulation comprising polyurethane-35 in combination with dicalcium phosphate.

### Example 8

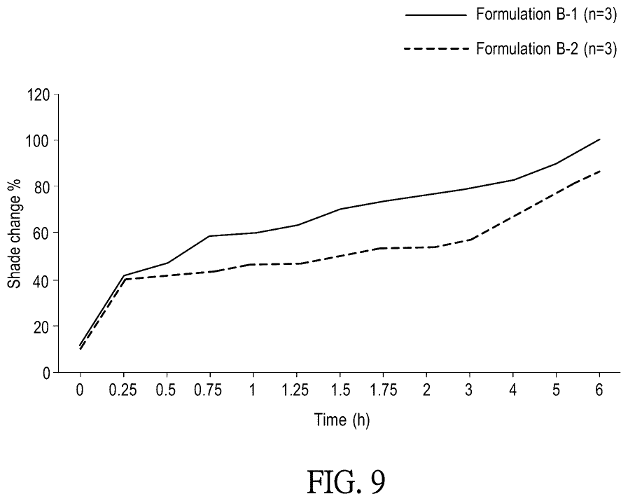

Example 8 was performed in a manner similar to Example 5 above. The anti-staining gel formulations B-1 and B-2 containing different concentrations of polyurethane-35, dicalcium phosphate and calcium lactate as shown in Table 9 were formulated respectively, and the rest of the ingredients were the same, as shown in Table 1A. The detail results of staining resistance were shown in FIG. 9.

TABLE 9

| Components | formulation B-1 | formulation B-2 |
|---|---|---|
| X | 15% | 15% |
| Y | 0% | 1% |
| Z | 0.5% | 0.5% |

The formulations B-2 and B-3 were formulated according to Example 2 above. The formulation B-1 comprised 15% polyurethane-35, 0% dicalcium phosphate and 0.5% calcium lactate. The average shade of the teeth of formulation B-1 group before being soaked in the staining solution was 2, which increased to 14.8 after 1 hour of continuous soaking, and the shade change was 51.03%, and increased to 18.3 after 2 hours of continuous soaking, and the shade change was 53.44%. After 6 hours of continuous soaking, the average shade of the teeth reached 25.7, and the shade change was 88.62%. The formulation B-2 comprised 15% polyurethane-35, 1% dicalcium phosphate and 0.5% calcium lactate. The average shade of the teeth of formulation B-2 group before being soaked in the staining solution was 3, which increased to 15.5 after 2 hours of continuous soaking, and the shade change was 53.44%, and increased to 25 after 6 hours of continuous soaking, and the shade change was 86.2%. The above results showed that the formulation comprising polyurethane-35 and dicalcium phosphat in combination with calcium lactate was more able to prolong the staining resistance of teeth than the formulation comprising polyurethane-35 in combination with calcium lactate.

### Correlation of Three Ingredients for Staining Resistance

{X}: representing the staining resistance of the formulation comprising only polyurethane-35 (expressed in % of shade change)
{Y}: representing the staining resistance of the formulation comprising only dicalcium phosphate (expressed in % of shade change)
{Z}: representing the staining resistance of the formulation comprising only calcium lactate (expressed in % of shade change)
{X+Y}: representing the staining resistance of the formulation comprising polyurethane-35 in combination with dicalcium phosphate (expressed in % of shade change)
{X+Z}: representing the staining resistance of the formulation comprising polyurethane-35 in combination with calcium lactate (expressed in % of shade change)
{X+Y+Z}: representing the staining resistance of the formulation comprising polyurethane-35 in combination with dicalcium phosphate and calcium lactate and (expressed in % of shade change)

From the results in FIG. 6-3, it can be seen that the combination of polyurethane-35 and dicalcium phosphate had at least a synergistic effect. The shade change rates of the formulations E and C-1 in the fourth hour were 80.45% and 67.24%, respectively, and the obtained relation was {X+Y}≥1.23{X}. From the results in FIG. 7, it can be seen that the combination of polyurethane-35 and calcium lactate had at least a synergistic effect. The shade change rates of the formulations E and B-1 in the fourth hour were 80.45% and 66.2%, respectively, and the obtained relation is {X+Z}≥1.03{X}. From the results in FIG. 8, it can be seen that the combination of polyurethane-35, dicalcium phosphate and calcium lactate had at least a synergistic effect. The shade change rates of the formulations D-2 and D-3 in the fourth hour were 46.66% and 43.21%, respectively, and the obtained relation was {X+Y+Z}≥1{X+Y}. From the results in FIG. 9, it can be seen that the combination of polyurethane-35, dicalcium phosphate and calcium lactate had at least a synergistic effect. The shade change rates of the formulations B-1 and B-2 in the fourth hour were 82.75% and 67.24%, respectively, and the obtained relation was {X+Y+Z}≥1.37{X+Z}. It can be seen from the above that the combined formulations in such concentration ranges had at least a synergistic effect on the staining resistance of teeth.

### Synergistic Effect of Three Ingredients for Staining Resistance

The interaction between the two reagents was evaluated with reference to the Combination Index (CI) of the two reagents proposed by T. C. Chou (Ting-Chao Chou. Drug Combination Studies and Their Synergy Quantification Using the Chou-Talalay Method. Cancer Res. 2010; 70(2): 440-446) and L. Zhao et al. (Liang Zhao, M. Guillaume Wientjes, Jessie L-S. Au. Evaluation of Combination Chemotherapy: Integration of Nonlinear Regression, Curve Shift, Isobologram, and Combination Index Analyses. Clinical Cancer Research 2004; vol. 10: 7994-8004). CI<1 means that the combination reagent has a synergistic effect; CI≈1 means that the combination reagent has an additive effect; and CI>1 means that the combination reagent has an antagonistic effect. The greater CI value represents a greater the possibility of antagonism.

### A. Synergistic Effect of Polyurethane-35 and Dicalcium Phosphate

The measurement time was 6 hours as the end point of the experiment. The time corresponding to the shade change rate of up to 70% in the different formulations was used as the calculated value, and the time for each concentration group to reach 70% change was defined as CI_{70%}. The formula was as follows:

$$C {I}_{70 \%} = \frac{6 - 4.2 ( h r )}{6 - 1.625 ( h r )} + \frac{6 - 4.2 ( h r )}{6 - 1.5 ( h r )} \approx 0.81 < 1$$

The calculated CI value was about 0.81, which confirmed that the combination of polyurethane-35 and dicalcium phosphate had a synergistic effect.

### B. Synergistic Effect of Polyurethane-35 and Calcium Lactate

The measurement time was 6 hours as the end point of the experiment. The time corresponding to the shade change rate of up to 70% in the different formulations was used as the calculated value, and the time for each concentration group to reach 70% change was defined as CI_{70%}. The formula was as follows:

$$C {I}_{70 \%} = \frac{6 - 4 ( h r )}{6 - 1.625 ( h r )} + \frac{6 - 4 ( h r )}{6 - 0.37 ( h r )} \approx 0.812 < 1$$

The calculated CI value was about 0.812, which confirmed that the combination of polyurethane-35 and calcium lactate had a synergistic effect.

### C. Synergistic Effect of Polyurethane-35, Dicalcium Phosphate and Calcium Lactate

The measurement time was 6 hours as the end point of the experiment. The time corresponding to the shade change rate of up to 45% in the different formulations was used as the calculated value due to the significant synergistic effect of the combination of the three ingredients, and the time for each concentration group to reach 45% change was defined as CI_{45%}.
I. Synergistic effect of polyurethane-35 in combination with dicalcium phosphate and calcium lactate

$$C {I}_{45 \%} = \frac{6 - 5.89 ( h r )}{6 - 0.89 ( h r )} + \frac{6 - 5.89 ( h r )}{6 - 0.16 ( h r )} \approx 0.04 < 1$$

The calculated CI value was about 0.04, which confirmed that the combination of polyurethane-35, dicalcium phosphate and calcium lactate had a synergistic effect.
II. Synergistic effect of polyurethane-35 in combination with calcium lactate and dicalcium phosphate

$$C {I}_{45 \%} = \frac{6 - 5.89 ( h r )}{6 - 0.54 ( h r )} + \frac{6 - 5.89 ( h r )}{6 - 0.2 ( h r )} \approx 0.04 < 1$$

The calculated CI value was about 0.04, which confirmed that the combination of polyurethane-35, calcium lactate and dicalcium phosphate had a synergistic effect.

Based on the above experimental results, it can be confirmed that polyurethane-35 in combination with dicalcium phosphate and polyurethane-35 in combination with calcium lactate had synergistic effects; and the combined formulation containing polyurethane-35, dicalcium phosphate and calcium lactate also had a synergistic effect.

### Example 9

The collected adult human molars were cleaned by ultrasonic vibration with 37% phosphoric acid and ultrapure water, and then soaked in a staining solution of concentrated black coffee at 37±2.5° C. for 48 hours to stain the teeth. Then, the teeth were soaked in different reagents according to the following manner, and each tooth shade was recorded by using a VITA tooth color chart and a camera (Canon M6 Mark ii).

The stained teeth were divided into Blank group, Whitening-no anti-stain treatment group and Whitening-anti-stain treatment group. Each group was tested with three teeth (N=3). The t-test was used for statistical analysis, and the t-test was calculated based on the group mean. Each group performs the following procedures:
(i) Blank group:
Every day, the teeth were soaked in ultrapure water for 25 minutes, rinsed with ultrapure water to simulate mouthwash, and put into PBS simulation medium to simulate the oral environment, and then incubated at 37° C.±2.5° C. for 24 hours. The above steps were repeated for 7 days.
(ii) Whitening-no anti-stain treatment group:
Every day, the teeth were soaked in whitening gel (refer to Table 1B) for 25 minutes, rinsed with ultrapure water, and put into PBS simulation medium and incubated at 37° C.±2.5° C. for 24 hours. The above steps were repeated for 7 days.
(iii) Whitening-anti-stain treatment group:
Every day, the teeth were soaked in whitening gel (refer to Table 1B) for 25 minutes, rinsed with ultrapure water, soaked in anti-staining gel (refer to Table 1A), and put into PBS simulation medium and incubated at 37° C.±2.5° C. for 24 hours. The above steps were repeated for 7 days.

Then, the teeth of Whitening-no anti-stain treatment group and Whitening-anti-stain treatment group were soaked in a staining solution of concentrated black coffee for 18 hours. The correlation values between shade changes of teeth and pigment accumulation time were observed every 15 minutes with VITA tooth color chart and a camera (Canon M6 Mark ii).

### Tooth Whitening Effect

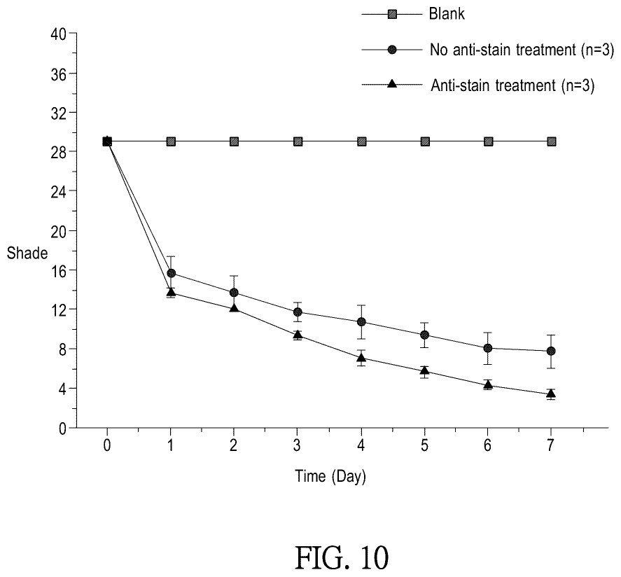

According to the above, the whitening test results of Blank group, Whitening-no anti-stain treatment group and Whitening-anti-stain treatment group for 7 days were shown in FIG. 10. It can be clearly seen from FIG. 10 that Blank group has no tooth whitening effect, while both Whitening-no anti-stain treatment group and Whitening-anti-stain treatment group have significant tooth whitening effects. During the 7-day test period, Whitening-no anti-stain treatment group and Whitening-anti-stain treatment group decreased an average of 21 and 25.6 shades, respectively, wherein the tooth whitening effect of Whitening-anti-stain treatment group was the best, which was 4.5 shades better than that of Whitening-no anti-stain treatment group.

### Tooth Anti-staining Effect

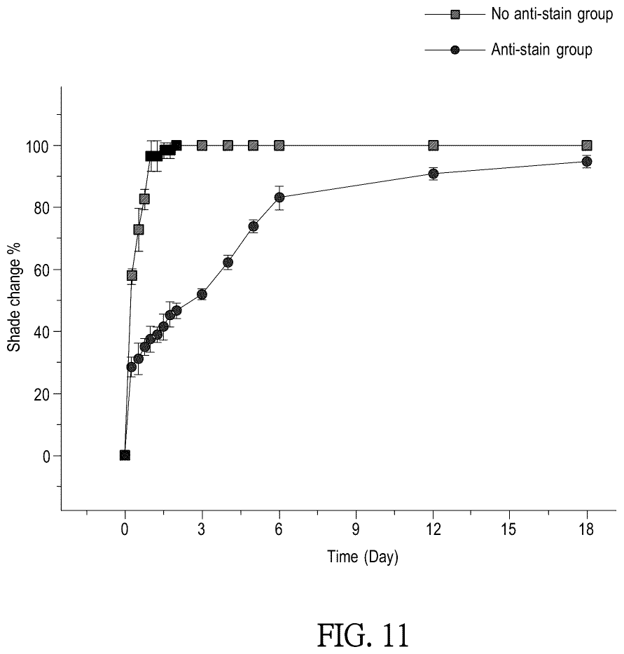

After a 7-day whitening test, Whitening-no anti-stain treatment group and Whitening-anti-stain treatment group were placed in a staining solution of black coffee to further analyze the anti-staining effect of the anti-staining gel. The results were shown in FIG. 11. It can be seen from FIG. 11 that in the interval of observation every 15 minutes, during the first two hours, the teeth of Whitening-no anti-stain treatment group were found to be less resistant to coffee pigment. After two hours, the shade of teeth rose from 9 to 29, and the color reversion rat reached 100%. Whitening-no anti-stain treatment group was resistant to coffee pigment. After two hours, the shade of teeth rose from 3 to 16, and the color reversion rat only reached 46.2%, and the color reversion rat only reached 80% after 6 hours. In contrast, the teeth treated with the anti-staining composition of the present disclosure can effectively enhance the anti-staining effect of the teeth on the caffeine pigment. In other words, the anti-staining composition of the present disclosure can prolong the whitening effect of the tooth whitening agent of the present disclosure, and can delay the adhesion of pigment after tooth whitening.

The results of the above examples show that the anti-staining effect of the teeth treated with the tooth anti-staining composition of the present disclosure is better than that of the teeth untreated with the tooth anti-staining composition of the present disclosure, and the anti-staining time of the teeth can be prolonged. In addition, based on the safety and the resistance of teeth to coffee staining, within a safe concentration, the tooth anti-staining composition containing 30% polyurethane-35 can most effectively improve the teeth's anti-staining effect against coffee pigment and last 6 hours. The tooth anti-staining composition containing 3% polyurethane-35 can still make the teeth have anti-staining effect on coffee pigment even after being soaked in coffee for six hours.

As shown in the results of anti-staining effect, the formulation comprising polyurethane-35 in combination with dicalcium phosphate or calcium lactate can more effectively improve the staining resistance of teeth in long-term or frequent coffee environment compared with the formulation comprising polyurethane-3. As shown in the results of FIGS. 8 and 9, the formulation comprising polyurethane-35 in combination with dicalcium phosphate and calcium lactate can more effectively improve the staining resistance of teeth in long-term or frequent coffee environment compared with the formulation comprising polyurethane-3 without dicalcium phosphate or calcium lactate. Even teeth soaked in coffee for 6 hours were still resistant to caffeine pigment.

Moreover, adding a certain proportion of dicalcium phosphate and/or calcium lactate to the anti-stain composition containing polyurethane-35 can synergistically assist polyurethane-35 to enhance the staining resistance of teeth, and can significantly improve the staining resistance of teeth in long-term.

### Enumerated Embodiments

The following exemplary embodiments are provided, the numbering of which is not to be construed as designating levels of importance.

Embodiment 1 provides a tooth anti-staining composition, which comprises 3% to 30% of polyurethane and 0.1% to 25% of dicalcium phosphate by weight of the tooth anti-staining composition and orally acceptable carriers and/or excipients.

Embodiment 2 provides the tooth anti-staining composition of Embodiment 1, which further comprises 0.1% to 25% of calcium lactate by weight of the tooth anti-staining composition.

Embodiment 3 provides the tooth anti-staining composition of Embodiment 1 or 2, which further comprises 2.5% to 30% of Polyvinylpyrrolidone, 0.2% to 20% of acrylates copolymer and/or 0.1% to 25% of titanium dioxide by the weight of the tooth anti-staining composition, and the total weight of all components in the tooth anti-staining composition is 100%.

Embodiment 4 provides the tooth anti-staining composition of any of Embodiments 1-3, which further comprises water.

Embodiment 5 provides the tooth anti-staining composition of any of Embodiments 1-3, which further comprises at least one humectant.

Embodiment 6 provides the tooth anti-staining composition of Embodiment 5, wherein the humectant is selected from the group consisting of sorbite, glycerol, xylitol, propylene glycol and the combination thereof.

Embodiment 7 provides the tooth anti-staining composition of any of Embodiments 1-3, which further comprises at least one surfactant.

Embodiment 8 provides the tooth anti-staining composition of Embodiment 7, wherein the surfactant is selected from the group consisting of polysorbate 20, polysorbate 40, polysorbate 60, polysorbate 65, polysorbate 80 and the combination thereof.

Embodiment 9 provides the tooth anti-staining composition of any of Embodiments 1-3, which further comprises at least one potassium salt of anti-sensitizer.

Embodiment 10 provides the tooth anti-staining composition of Embodiment 10, wherein the potassium salt of anti-sensitizer is selected from the group consisting of potassium citrate, potassium nitrate, potassium chloride and the combination thereof.

Embodiment 11 provides the tooth anti-staining composition of any of Embodiments 1-3, which further comprises at least one anti-bacterial or preservative.

Embodiment 12 provides the tooth anti-staining composition of Embodiment 11, wherein the anti-bacterial or preservative is selected from the group consisting of phenoxyethanol, benzoic acid, salicylic acid, p-hydroxybenzoic acid, methyl-p-hydroxybenzoate, chlorphenesin, ethylhexylglycerin and the combination thereof.

Embodiment 13 provides the tooth anti-staining composition of any of Embodiments 1-3, which further comprises at least one flavoring agent.

Embodiment 14 provides the tooth anti-staining composition of Embodiment 13, wherein the flavoring agent includes essential oils and various flavoring aldehydes, esters and alcohols, wherein the example of the essential oil includes an oil extract from lemon, lime, grapefruit, orange, peppermint, wintergreen, sassafras, clove, sage, spearmint, marjoram or cinnamon.

Embodiment 15 provides the tooth anti-staining composition of Embodiment 13, wherein the flavoring agent includes acesulfame potassium, ethyl menthane carboxamide, menthol, carvone or anethole.

Embodiment 16 provides the tooth anti-staining composition of any of Embodiments 1-3, which further comprises at least one abrasive.

Embodiment 17 provides the tooth anti-staining composition of Embodiment 17, wherein the abrasive is selected from the group consisting of aluminum hydroxide, aluminum silicate, dicalcium phosphate, silica, calcium carbonate, bentonite and the combination thereof.

Embodiment 18 provides the tooth anti-staining composition of any of Embodiments 1-17, which is in a dosage form of a solution, slurry, gel, paste or ointment.

Embodiment 19 provides a tooth anti-staining kit, comprising the tooth anti-staining composition of any of Embodiments 1-17 and an instruction for use.

Embodiment 20 provides a tooth whitening and anti-staining kit, comprising the tooth anti-staining composition of any of Embodiments 1-17, a tooth whitening composition and an instruction for use.

Embodiment 21 provides a method for anti-staining of teeth, comprising applying the tooth anti-staining composition of any of Embodiments 1-17 on the dental surfaces for a period of time, and then rinsing with water to remove the tooth anti-staining composition that is not adhered on the tooth surface.

Embodiment 22 provides the method of Embodiment 21, wherein the teeth have been whitened by a tooth whitening agent prior to applying the tooth anti-staining composition.

Embodiment 23 provides the method of Embodiment 21, wherein the teeth have been cleaned with a dentifrice prior to applying the tooth anti-staining composition.

What is claimed is:

1. A tooth anti-staining composition, comprising 3% to 30% of polyurethane and 0.1% to 25% of dicalcium phosphate by weight of the tooth anti-staining composition and orally acceptable carriers and/or excipients.

2. The tooth anti-staining composition of claim 1, further comprising 0.1% to 25% of calcium lactate by weight of the tooth anti-staining composition.

3. The tooth anti-staining composition of claim 1, further comprising 2.5% to 30% of Polyvinylpyrrolidone, 0.2% to 20% of acrylates copolymer and/or 0.1% to 25% of titanium dioxide by the weight of the tooth anti-staining composition, and the total weight of all components in the tooth anti-staining composition is 100%.

4. The tooth anti-staining composition of claim 1, wherein the orally acceptable carriers and/or excipients are one or more selected from the group consisting of a humectant, a surfactant, an anti-sensitizer, an anti-bacterial or a preservative, a flavoring agent and an abrasive.

5. The tooth anti-staining composition of claim 4, wherein the humectant is selected from the group consisting of water, sorbate, glycerol, xylitol, propylene glycol and the combination thereof.

6. The tooth anti-staining composition of claim 4, wherein the surfactant is selected from the group consisting of polysorbate 20, polysorbate 40, polysorbate 60, polysorbate 65, polysorbate 80 and the combination thereof.

7. The tooth anti-staining composition of claim 4, wherein the anti-sensitizer is selected from the group consisting of potassium citrate, potassium nitrate, potassium chloride and the combination thereof.

8. The tooth anti-staining composition of claim 4, wherein the anti-bacterial or preservative is selected from the group consisting of phenoxyethanol, benzoic acid, salicylic acid, p-hydroxybenzoic acid, methyl-p-hydroxybenzoate, Chlorphenesin, ethylhexylglycerin and the combination thereof.

9. The tooth anti-staining composition of claim 4, wherein the flavoring agent is selected from the group consisting of lemon oil, lime oil, grapefruit oil, orange oil, peppermint oil, wintergreen oil, sassafras oil, clove oil, sage oil, spearmint oil, marjoram oil, cinnamon oil, acesulfame potassium, ethyl menthane carboxamide, menthol, carvone, anethole and the combination thereof.

10. The tooth anti-staining composition of claim 4, wherein the abrasive is selected from the group consisting of aluminum hydroxide, aluminum silicate, dicalcium phosphate, silica, calcium carbonate, bentonite and the combination thereof.

11. The tooth anti-staining composition of claim 1, which is in a dosage form of a solution, slurry, gel, paste or ointment.

12. A tooth anti-staining kit, comprising the tooth anti-staining composition of claim 1 and an instruction for use.

13. A tooth whitening and anti-staining kit, comprising the tooth anti-staining composition of claim 1, a tooth whitening composition and an instruction for use.

14. A method for anti-staining of teeth, comprising applying the tooth anti-staining composition of claim 1 on the dental surfaces for a period of time, and then rinsing with water to remove the tooth anti-staining composition that is not adhered on the tooth surface.

15. The method of claim 14, wherein the teeth have been whitened by a tooth whitening agent prior to applying the tooth anti-staining composition.

16. The method of claim 14, wherein the teeth have been cleaned with a dentifrice prior to applying the tooth anti-staining composition.
